(19) United States
(12) Patent Application Publication
(10) Pub. No.: US 2023/0260661 A1
(43) Pub. Date: Aug. 17, 2023

(54) SYSTEM AND METHOD FOR AUGMENTING ANEURYSM LEARNING DATA

(71) Applicants: INDUSTRY-UNIVERSITY COOPERATION FOUNDATION HANYANG UNIVERSITY ERICA CAMPUS, Ansan-si (KR); INDUSTRY-ACADEMIC COOPERATION FOUNDATION, YONSEI UNIVERSITY, Seoul (KR)

(72) Inventors: Hyeondong YANG, Geoje-si (KR); Je Hoon OH, Anyang-si (KR); Yong Bae KIM, Seoul (KR); Kwang-Chun CHO, Seoul (KR); Jung-Jae KIM, Seoul (KR)

(73) Assignees: INDUSTRY-UNIVERSITY COOPERATION FOUNDATION HANYANG UNIVERSITY ERICA CAMPUS, Ansan-si (KR); INDUSTRY-ACADEMIC COOPERATION FOUNDATION, YONSEI UNIVERSITY, Seoul (KR)

(21) Appl. No.: 17/928,777

(22) PCT Filed: Jun. 23, 2021
(86) PCT No.: PCT/KR2021/007902

§ 371 Date: Nov. 30, 2022

(30) Foreign Application Priority Data
Jun. 24, 2020 (KR) 10-2020-0077206
Jun. 24, 2020 (KR) 10-2020-0077309
Jun. 24, 2020 (KR) 10-2020-0077368

(51) Int. Cl. G16H 50/50; G16H 30/20; G06T 7/00

(52) U.S. Cl. CPC G16H 50/50; G06T 7/0012; G16H 30/20; G06T 2207/30104

(57) ABSTRACT

The present invention relates to a method and system for augmenting aneurysm learning data for augmenting artificial images formed of various result values calculated from simulation results. The method of augmenting aneurysm learning data according to the present invention includes: performing a simulation using aneurysm data; predicting a position having a smallest thickness in an aneurysm based on a result of the simulation; setting a center position at the predicted position; setting a plurality of peripheral positions at different positions having a preset radius from the center position; extracting blood flow data according to a preset sampling period for a reference time at each of the center position and the plurality of peripheral positions; converting the extracted blood flow data into an image to generate a blood flow image; and generating a central image and a peripheral image in which a plurality of blood flow images according to the center position and the peripheral position are arranged in the order of the reference time; and generating different artificial images by changing an arrangement order of the central image and the peripheral image.

### TECHNICAL FIELD

The present invention relates to a method and system for augmenting aneurysm learning data, and more particularly, to a method and system for augmenting aneurysm learning data that are capable of augmenting an artificial image formed of various result values calculated from simulation results.

### BACKGROUND ART

In general, diseases of the circulatory system include thickening, hardening, stenosis, and the like of blood vessels. These diseases are lesions in normal areas caused by an influence of blood flow, and there are cases where subsequent progression of the diseases may lead to death, and there is a problem that treatment methods of the diseases may be life-threatening. To resolve such intractable circulatory system diseases, it is useful to use engineering techniques such as fluid analysis and structural analysis based on pathological fragments.

For example, a cerebral aneurysm is a vascular disorder in which a part of a cerebral artery wall protrudes outwardly in a bag shape, but the number of cases that are accidentally discovered in an unruptured state when a brain is image-diagnosed is increasing. The cerebral aneurysm is due to fragility of an artery wall, etc., and refers to the swelling of a blood vessel wall in a shape of a lump. The cerebral aneurysm does not have a media, and therefore, is easily destroyed, and many cerebral aneurysms are present in the subarachnoid space and are therefore the largest cause of subarachnoid hemorrhage. Therefore, appropriate preventive treatments such as clipping with craniotomy, coil embolization, and stent treatment needs to be performed on the cerebral aneurysm.

Although these treatments require a very high level of proficiency, it is difficult for many medical staffs to access these related procedures. In addition, since blood vessel shapes are different for each individual, even when the same treatment method is performed, a detailed treatment plan may be changed according to the difference in shape. Accordingly, recently, treatment plans and measures have been established, but since the prognosis of an aneurysm cannot be predicted only with a shape of a simple aneurysm, a hemodynamic simulation is being applied.

However, there is a problem in that the aneurysm simulation takes a lot of time to derive the results, and the more complex the simulation model and the larger the analysis process, the more time is consumed.

The present invention was developed with the support of 1) first project (Project name: Analysis of cerebral aneurysm formation mechanism and development of rupture risk prediction model based on hemodynamics and histopathology, Research Project Identification Number: 2021-03-0296, Project Identification Number (Government): 2020R1A2C1011918, Department in charge of Business: Ministry of Science and ICT, Supervision Institution: National Research Foundation of Korea, Research Period: Mar. 1, 2021 to Feb. 28, 2022), and 2) second project (Project name: Energy harvesting/storage device, development of multi-material-based high-reliability wearable system in which sensor and actuator are integrated, Research Project Identification Number: 202100000001065, Project Identification Number (Government): 2019R1A2C1005023, Department in charge of Business: Ministry of Science and ICT (2017Y), Supervision Institution: National Research Foundation of Korea, Research Period: Mar. 1, 2021 to Feb. 28, 2022).

### Technical Problem

The technical object of the present invention is to solve this problem, and to provide a method and system for augmenting aneurysm learning data that are capable of augmenting an artificial image formed of various result values calculated from simulation results.

The technical problems of the present invention are not limited to the above-mentioned aspects, and other technical problems that are not described may be obviously understood by those skilled in the art from the following description.

### Technical Solution

According to an aspect of the present invention, a method of augmenting aneurysm learning data includes performing a simulation using aneurysm data; predicting a position having a smallest thickness in an aneurysm based on a result of the simulation; setting a center position at the predicted position; setting a plurality of peripheral positions at different positions having a preset radius from the center position; extracting blood flow data according to a preset sampling period for a reference time at each of the center position and the plurality of peripheral positions; converting the extracted blood flow data into an image to generate a blood flow image; and generating a central image and a peripheral image in which a plurality of blood flow images according to the center position and the peripheral position are arranged in the order of the reference time; and generating different artificial images by changing an arrangement order of the central image and the peripheral image.

The method may further include resetting a new peripheral position, which is different from the plurality of peripheral positions, from the center position.

The resetting of the peripheral position may include setting at least one of a rotation direction from the center position, a rotation angle from the center position, the number of peripheral positions, and the radius to be different to set a new peripheral position located at a different position.

The blood flow data may include at least one of blood flow velocity, pressure, a strain rate, a deformation amount, stress, force, wall shear stress (WSS), and an oscillatory shear index (OSI).

In the generating of the artificial image, a new artificial image may be generated by combining the different artificial images according to different blood flow data.

A size of the radius may be changeable according to a size of the aneurysm.

A size of the radius may be changeable according to a degree of complexity of a shape of the aneurysm.

According to another aspect of the present invention, a method of augmenting aneurysm learning data includes: performing a simulation using aneurysm data; predicting a position having a smallest thickness in an aneurysm based on a result of the simulation; setting a center position at the predicted position; setting at least one of a rotation direction from the center position, a rotation angle from the center position, a radius from the center position, and a number to be different to set a plurality of peripheral positions spaced an equal distance from the center position in different directions; extracting blood flow data according to a preset sampling period for a reference time at each of the center position and the plurality of peripheral positions; converting the extracted blood flow data into an image to generate a blood flow image; generating a central image and a peripheral image in which a plurality of blood flow images according to the center position and the peripheral position are arranged in the order of the reference time; and generating an artificial image from the central image and the peripheral image.

The method may further include, after the generating of the central image and the peripheral image, generating a new artificial image by changing an arrangement order of the central image and the peripheral image.

According to still another aspect of the present invention, a system for augmenting aneurysm learning data includes: a simulation module configured to perform a simulation using aneurysm data; a positioning module configured to predict a position having a smallest thickness in an aneurysm based on a result of the simulation to set a center position at the predicted position and a plurality of peripheral positions at different positions having a preset radius from the center position; a data extraction module configured to extract blood flow data according to a preset sampling period for a reference time at each of the center position and the plurality of peripheral positions; and an artificial image generation module configured to convert the extracted blood flow data into an image to generate an artificial image.

The positioning module may reset a new peripheral position, which is different from the plurality of peripheral positions, from the center position.

The artificial image generation module may convert the blood flow data of the center position and a new peripheral position into an image to generate a blood flow image, and combine a central image and a peripheral image in which a plurality of blood flow images according to the center position and the peripheral position are arranged in the order of the reference time to generate the artificial image.

Different artificial images may be repeatedly generated by changing an arrangement order of the central image and the peripheral image.

The positioning module may set a rotation direction from the center position, a rotation angle from the center position, the number of peripheral positions, the radius, and the like to be different to set the new peripheral position located at a different position.

The blood flow data may include at least one of blood flow velocity, pressure, a strain rate, a deformation amount, stress, force, WSS, and an OSI.

The artificial image generation module may generate a new artificial image by combining the different artificial images according to different blood flow data.

A size of the radius may be changeable according to a size of the aneurysm.

A size of the radius may be changeable according to a degree of complexity of a shape of the aneurysm.

### Advantageous Effects

According to a method and system for augmenting aneurysm learning data of the present invention, it is possible to generate a large amount of data based on a result value calculated by performing a simulation. In addition, according to the method and system for augmenting aneurysm learning data of the present invention, it is possible to change an arrangement order of result values calculated through a simulation and generate a large number of artificial images from the arrangement order.

### DESCRIPTION OF DRAWINGS

FIG. 1 is a configuration diagram of a system for augmenting aneurysm learning data of the present invention.

FIG. 2 is a flowchart of a method of augmenting aneurysm learning data of the present invention.

FIGS. 3 to 12 are diagrams illustrating a process of augmenting aneurysm learning data.

FIG. 13 is a graph showing a deep learning result to which the method of augmenting aneurysm learning data of the present invention is applied.

FIG. 14 is a configuration diagram of an aneurysm prediction system using a deep learning algorithm of the present invention.

FIG. 15 is a flowchart of an aneurysm prediction method using the deep learning algorithm of the present invention.

FIG. 16 is a flowchart of a simulation-based deep learning algorithm.

FIG. 17 is a diagram to which various aneurysm simulations are applied.

FIG. 18 is a diagram illustrating a construction model according to the application of various aneurysm simulations.

FIG. 19 is a diagram to which various pieces of blood flow data is applied.

FIG. 20 is a diagram illustrating a treatment method predicted through the aneurysm prediction method using the deep learning algorithm.

FIG. 21 is a cross-sectional view of a blood vessel for practice of the present invention.

FIG. 22 is a diagram illustrating a blood vessel model.

FIG. 23 is a cross-sectional view of a blood vessel for practice illustrating a modified example of the auxiliary outer layer and an aneurysm inducing layer.

FIG. 24 is a photograph illustrating a manufacturing procedure of the blood vessel for practice of the present invention.

FIG. 25 is a diagram illustrating a method of manufacturing a blood vessel for practice according to the present invention.

FIG. 26 is a flowchart of a method of manufacturing a blood vessel for practice.

FIGS. 27 and 28 are diagrams illustrating a blood vessel for practice according to another embodiment.

FIG. 29 is a diagram illustrating a method of manufacturing a blood vessel for practice according to another embodiment.

FIG. 30 is a flowchart of a method of manufacturing a blood vessel for practice according to another embodiment.

### MODES OF THE INVENTION

Various advantages and features of the present invention and methods accomplishing them will become apparent from the following description of embodiments with reference to the accompanying drawings. However, the present invention is not limited to exemplary embodiments to be described below, but may be implemented in various different forms, these exemplary embodiments will be provided only in order to make the present invention complete and allow those skilled in the art to completely recognize the scope of the present invention, and the present invention will be defined by the scope of the claims. Throughout the specification, like reference numerals denote like elements.

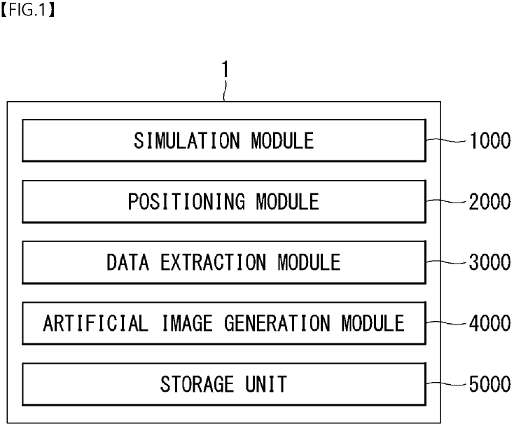

Hereinafter, a method and system for augmenting aneurysm learning data of the present invention will be described with reference to FIGS. 1 to 13.

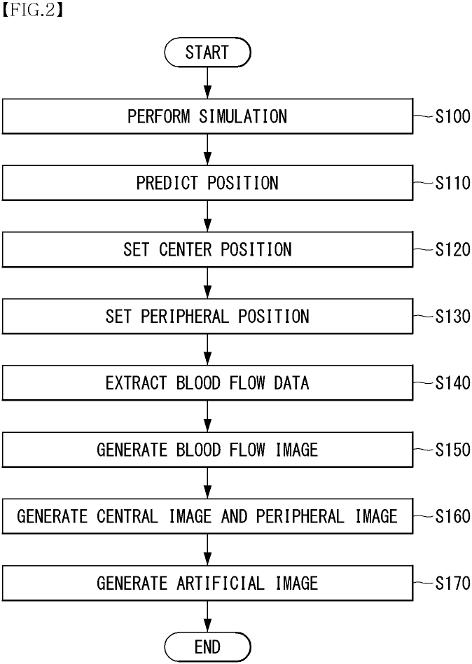

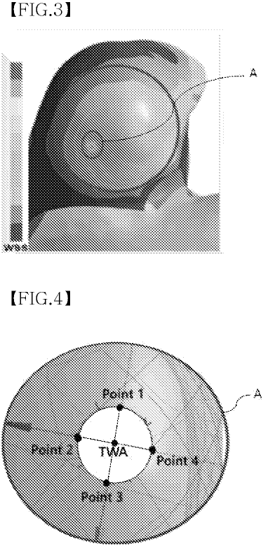

After describing the system for augmenting aneurysm learning data of the present invention with reference to FIG. 1, the method of augmenting aneurysm learning data of the present invention will be described with reference to FIGS. 2 to 13.

Hereinafter, the system for augmenting aneurysm learning data of the present invention will be described with reference to FIG. 1.

FIG. 1 is a configuration diagram of a system for augmenting aneurysm learning data of the present invention.

Referring to FIG. 1, the system 1 for augmenting aneurysm learning data of the present invention is for generating various pieces of learning data from simulation results to which aneurysm data is applied, and may generate a large amount of data based on a result value calculated by performing a simulation. According to the method and system for augmenting aneurysm learning data of the present invention, it is possible to change an arrangement order of result values calculated through a simulation and generate a large number of artificial images from the arrangement order.

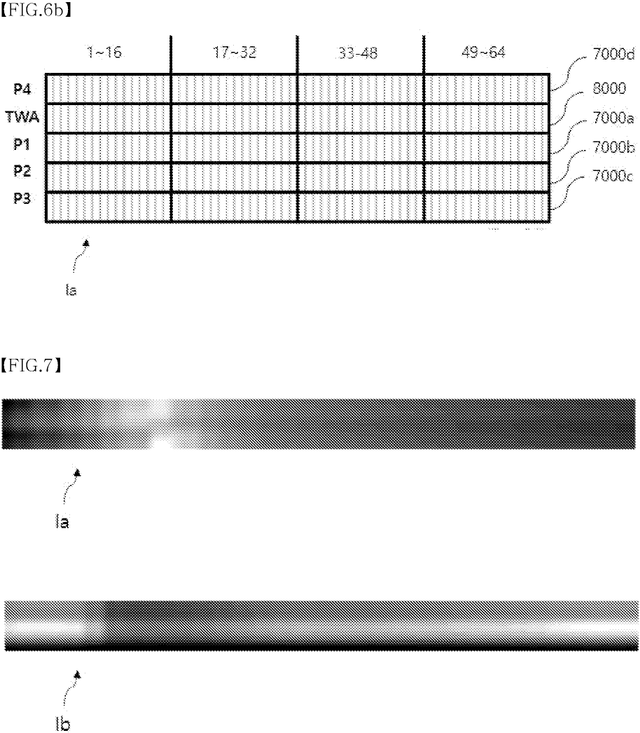

The system 1 for augmenting aneurysm learning data of the present invention includes a simulation module 1000, a positioning module 2000, a data extraction module 3000, and an artificial image generation module 4000. Specifically, the system 1 for augmenting aneurysm learning data includes a simulation module 1000 that performs a simulation using aneurysm data, a positioning module 2000 that predicts the position of the smallest thickness in the aneurysm based on a simulation result and sets a center position T (see TWA in FIG. 4) at the predicted position and a plurality of peripheral positions P (see point in FIG. 4) at different positions having a predetermined radius from the center position T, and an artificial image generation module 4000 that converts blood flow data (see 70 in FIG. 4) according to a preset sampling period (see 60 in FIG. 4) into an image for a reference time at the center position T and the plurality of peripheral positions P to generate an artificial image (see 100 in FIG. 7).

The simulation module 1000 may simulate aneurysm data of an object to form learning data. The simulation result derived from the simulation module 1000 is stored in a storage unit 5000, and thus, may generate an image according to the simulation result through the positioning module 2000, the data extraction module 3000, the artificial image generation module 4000, etc.

The positioning module 2000 is a module for setting the center position T and the peripheral position P based on a simulation result, and may predict a position having the smallest thickness in the aneurysm based on the simulation result. The positioning module 2000 sets the center position T at the position with the smallest thickness in the aneurysm, and sets a plurality of peripheral positions P at different positions having a predetermined radius from the center position T.

The center position T and the peripheral positions P are positions at which blood flow data for generating an artificial image I are extracted, and may be set by the positioning module 2000 based on the simulation result. The center position T is set at the position having the smallest thickness in the aneurysm, and the peripheral positions P may be set at positions having the same radius from the center position T and set at different positions to surround the center position T, including a plurality of pieces. In this case, a size of the radius may be changed according to a size of the aneurysm or a degree of complexity of a shape of an aneurysm. In addition, the peripheral position P is a position that is initially set, and a new peripheral position P located at a different position may be formed by a rotation direction from the center position T, a rotation angle from the center position T, the number of peripheral positions P, etc., in addition to a radius. This will be described below in detail through the method of augmenting aneurysm learning data.

The data extraction module 3000 may extract the blood flow data at each of the center position T and the peripheral position P set at the positions predicted by the positioning module 2000.

The data extraction module 3000 may extract the blood flow data according to a preset sampling period 6000 for a reference time at each of the center position T and the plurality of peripheral positions P, and extract at least one of blood flow velocity, pressure, a strain rate, a deformation amount, stress, force, wall shear stress (WSS), and an oscillatory shear index (OSI). The data extraction module 3000 may extract the blood flow data for the preset reference time at the center position T and the peripheral position P. The reference time is a preset time, and may include a plurality of divided sampling periods 6000. Describing by way of example, when it is assumed that the preset time is 1 second and 1 second is divided by 64, the reference time may be set to 1 and the sampling period 6000 may be set to 1/64. Accordingly, the data extraction module 3000 may extract the blood flow data corresponding to 64 sampling cycles 6000 at each of the center position and the peripheral position for the reference time of 1 second, which will be described below in detail with reference to FIGS. 5 to 8.

The blood flow data extracted from the data extraction module 3000 may be converted into the artificial image I by the artificial image generation module 4000.

The artificial image generation module 4000 is a module for generating the artificial image I by converting a plurality of pieces of blood flow data extracted from the blood flow data extraction module 3000 into images. The artificial image generation module 4000 may convert the blood flow data into an image to generate a blood flow image, and arrange a plurality of blood flow images according to the center position T and the peripheral position P in a reference time order to generate a central image 8000 and a peripheral image 7000.

The artificial image generation module 4000 may convert the blood flow data according to the center position T and the peripheral position P into images to generate each of a blood flow image according to the center position T and a blood flow image according to the peripheral position P. The blood flow image according to the center position T is an image according to the sampling period 6000, and may be arranged in a reference time order to generate the central image 8000 according to the center position T. The blood flow image according to the peripheral position P is also an image according to the sampling period 6000, and may be arranged in the reference time order to generate the peripheral image 7000 according to the peripheral position P. The artificial image generation module 4000 converts the blood flow data extracted from the center position T and the peripheral position P into images to generate each of the central image 8000 and the peripheral image 7000, and lists the central image 8000 and the peripheral image 7000 to generate the artificial image I. Also, the artificial image generation module 4000 may change the arrangement order of the central image 8000 and the peripheral image 7000 to generate a plurality of artificial images I having different arrangement orders. The artificial image generation module 4000 may convert new blood flow data according to a new peripheral position P as well as the center position T and the peripheral position P into images to generate new blood flow image, arrange the central image 8000 and a new peripheral image, and change the arrangement order to generate a plurality of new artificial images.

As the simulation results extracted from the simulation module 1000, the positioning module 2000, the data extraction module 3000, and the artificial image generation module 4000, the center position T and the peripheral position P, the blood flow data at the center position T and the peripheral position P, the central image 8000 and the peripheral image 7000, the artificial image I, etc., may be stored in the storage unit 5000.

The storage unit 5000 is a memory that stores the simulation results extracted from the simulation module 1000, the positioning module 2000, the data extraction module 3000, and the artificial image generation module 4000, the center position T, the peripheral position P, the blood flow data, the blood flow image, the central image 8000, the peripheral image 7000, the artificial image I, etc., and may include a non-volatile memory such as a read only memory (ROM), a high-speed random access memory (RAM), a magnetic disk storage device, and a flash memory device, or other non-volatile semiconductor memory device. For example, the memory is a semiconductor memory device, and a secure digital (SD) memory card, a secure digital high capacity (SDHC) memory card, a mini SD memory card, a mini SDHC memory card, a trans flash (TF) memory card, a micro SD memory card, a micro SDHC memory card, a memory stick, a compact flash (CF), a multi-media card (MMC), MMC micro, an eXtreme digital (XD) card, etc., may be used. In addition, the memory may also include a network attached storage device that is accessed via a network.

Also, the storage unit 5000 may include a central processing unit (CPU), a graphic processing unit (GPU), and various types of storage devices that are implemented with a microprocessor, etc. These devices may be provided on an embedded printed circuit board (PCB).

The simulation module 1000, the positioning module 2000, the data extraction module 3000, the artificial image generation module 4000, etc., function as a central processing unit, the type of central processing unit may include a microprocessor, and the microprocessor may include a processing device in which an arithmetic logic operator, a register, a program counter, an instruction decoder, or a control circuit are provided on at least one silicon chip.

In addition, the microprocessor may include a GPU for graphic processing of an image or video. The microprocessor may be implemented in the form of a system on chip (SoC) including a core and a GPU. The microprocessor may include a single core, a dual core, a triple core, a quad core, or a multiple-number core thereof.

In addition, the simulation module 1000, the positioning module 2000, the data extraction module 3000, the artificial image generation module 4000, etc., may include a graphic processing board that includes a GPU, a RAM, or a ROM provided on a separate circuit board electrically connected to the microprocessor.

Hereinafter, a method of augmenting aneurysm learning data of the present invention will be described with eference to FIGS. 2 to 13.

FIG. 2 is a flowchart of the method of augmenting aneurysm learning data of the present invention, FIGS. 3 to 12 are diagrams illustrating a process of augmenting aneurysm learning data, and FIG. 13 is a graph showing a deep learning result to which the method of augmenting aneurysm learning data of the present invention is applied.

Referring to FIG. 2, the method of augmenting aneurysm learning data of the present invention includes performing a simulation (S100), predicting a position (S110), setting a center position T (S120), setting a peripheral position P (S130), extracting blood flow data (S140), generating a blood flow image (S150), generating the central image 8000 and the peripheral image 7000 (S160), and generating the artificial image I (S170).

Specifically, the method of augmenting aneurysm learning data according to the present invention includes performing a simulation using aneurysm data (S100), predicting a position having a smallest thickness in the aneurysm based on the simulation result (S110), setting a center position at the predicted position (S120), setting a plurality of peripheral positions at different positions having a preset radius from the center position T (S130), extracting blood flow data according to a preset sampling period 6000 for a reference time at each of the center position T and the plurality of peripheral positions P (S140), converting the extracted blood flow data into an image to generate a blood flow image (S150), and generating the central image 8000 and the peripheral image 7000 in which a plurality of blood flow images according to the center position T and the peripheral position P are arranged in a reference time order (S160); and generating the artificial image I from the central image 8000 and the peripheral image 7000 (S170).

In the operation of setting the peripheral position P, the plurality of peripheral positions P may be set at different positions having a preset radius from the center position T, but at least one of the rotation direction from the center position T, the rotation angle from the center position T, the radius from the center position T, and a number may be set differently to set the plurality of peripheral positions P spaced an equal distance from the center position T in different directions.

In addition, in the operation of generating the artificial image I, the artificial image I may also be generated from the central image 8000 and the peripheral image 7000, but different artificial images I may also be generated by changing the arrangement order of the central image 8000 and the peripheral image 7000.

In order to augment the aneurysm learning data, the simulation module 1000 performs a simulation using aneurysm data (S100). When the simulation is performed using the aneurysm data, shapes of an aneurysm having different colors may be viewed as illustrated in FIG. 3. In this case, part A in FIG. 3 indicates the position having the smallest thickness in the aneurysm, and the position having the smallest thickness in the aneurysm may be predicted by checking the simulation result (S110). The center position T may be set at the predicted position of the thinnest position in the aneurysm based on the simulation result (S120).

FIG. 4 is an enlarged view of part A of FIG. 3, and illustrates that the center position T and the peripheral position P are set. The positioning module 2000 sets, based on the simulation result, the center position T at point TWA, which is a point, at which the thickness is predicted to be thinnest in the aneurysm (S120). After setting the center position T, the plurality of peripheral positions P are set at different positions having a preset radius from the center position T (S130). The peripheral position P may be set at a point which is a position having a preset radius R from the center position T. The peripheral position P is set at a position having the same radius from the center position T, and is set to surround the center position T as set at Point 1 to Point 4 points illustrated in the drawing. The size of the radius of the peripheral position P may be changed and set according to the size of the aneurysm or the degree of complexity of the shape of the aneurysm. In the present specification, the setting of the peripheral position P at four points that are Point 1 to Point 4 is described by way of example, but the present invention is not limited thereto, and the number of peripheral positions P may vary in the operation of setting the peripheral position P. Although the center position T and the peripheral position P are described as being set to form a spherical shape at the TWA point and Points 1 to 4, the center position T and the peripheral position P may be formed to form a three-dimensional model other than the spherical shape. For example, the peripheral position P may form a three-dimensional shape such as a sphere, a rectangular parallelepiped, or a polyhedron surrounding the center position T, and information may be extracted from the three-dimensional model. In addition, when viewed from one side as in FIG. 4, the plurality of peripheral positions P surrounding the center position T are formed to have not only a circular shape but also a rectangular or polygonal shape, and aneurysm data may be extracted therefrom. In this specification, an example in which the plurality of peripheral positions P are formed to have the same radius, which is a distance from the center position T, is described, but the distance from the center position T may not be formed equally.

After the center position T and the peripheral position P are set, the blood flow data according to the preset sampling period 6000 is extracted at each of the center position T and the plurality of peripheral positions P for the reference time.

Referring to FIG. 5, FIG. 5 illustrates extracting blood flow data according to each time at the center position T and the peripheral position P by dividing 1 second by 64. The data extraction module 3000 extracts the blood flow data according to the preset sampling period 6000 for the reference time at each of the center position T and the plurality of peripheral positions P (S140). Assuming that the preset reference time is set to 1 and 1 is divided by 64, the reference time may be set to 1 second and the sampling period 6000 may be set to 1/64. Accordingly, the data extraction module 3000 may extract the blood flow data according to 64 sampling periods 6000 for 1 second at the center position T, and extract the blood flow data according to 64 sampling periods 6000 for 1 second at the peripheral position P. The data extraction module 3000 may extract blood flow data corresponding to 64 sampling periods 6000 at each of the center position T and the peripheral position P for the reference time of 1 second.

The blood flow image is generated by converting the blood flow data extracted from the center position T and the peripheral position P into an image (S150). The blood flow image is an image according to each sampling period 6000 extracted from the center position T and the peripheral position P, and 64 images may be extracted at each of the center position T and the peripheral position P.

The blood flow images according to the center position T and the peripheral position P generate each of the central image 8000 and the peripheral image 7000 in which the sampling period 6000 is arranged in the reference time order such as

$$\frac{1}{64} , \frac{2}{64} , \frac{3}{64} , … , \frac{63}{64} , \frac{64}{64}$$

(S160). The central image 8000 and the peripheral image 7000 are arranged as in FIG. 5 to form the artificial image I (S170). The artificial image I is generated by arranging the central image 8000 and four peripheral images 7000 regularly or changing the arrangement order. For example, as illustrated in FIG. 5, the central image 8000 and the peripheral image 7000 are regularly arranged in the order of Point 1, Point 2, Point 3, Point 4, and TWA to generate the artificial image I.

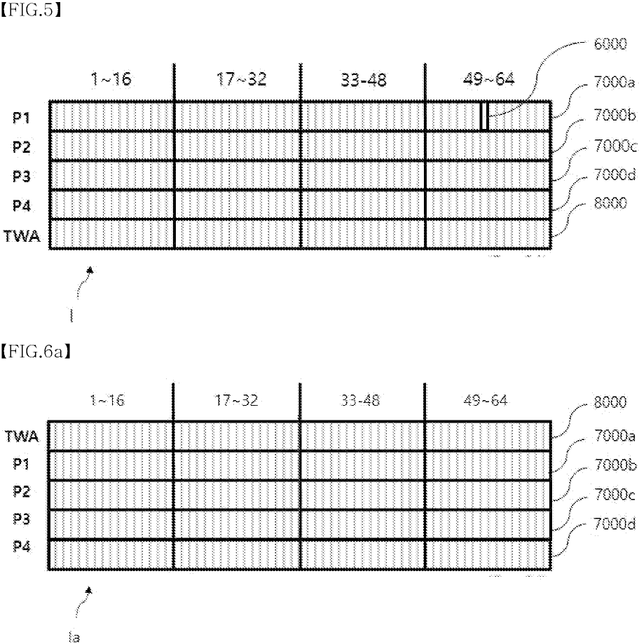

In addition, as illustrated in FIGS. 6a and 6b, by arranging the central image 8000 and the peripheral image 7000 in the order of TWA, Point 1, Point 2, Point 3, and Point 4 or in the order of Point 4, TWA, Point 1, Point 2, and Point 3, the arrangement order may be changed to generate a plurality of different artificial images 100a and 100b illustrated in FIG. 7, and different artificial images may also be generated by changing the arrangement order without being limited to the illustrated drawings.

As described above, the method of augmenting aneurysm learning data of the present invention can generate a plurality of images by changing the arrangement order of the central image 8000 and the peripheral image 7000. However, the present invention is not limited thereto, and a new artificial image may be generated by resetting the peripheral position P to a new peripheral position to form a new peripheral image according to the new peripheral position P.

The present invention may include resetting a new peripheral position P different from a plurality of peripheral positions P from the center position T. In the operation of resetting the peripheral position P, a new peripheral position P positioned at a different position may be set by differently setting at least one of the rotation direction from the center position T, the rotation angle from the center position T, the number of peripheral positions P, and the size of the radius, so it is possible to generate a new artificial image containing a new peripheral position P.

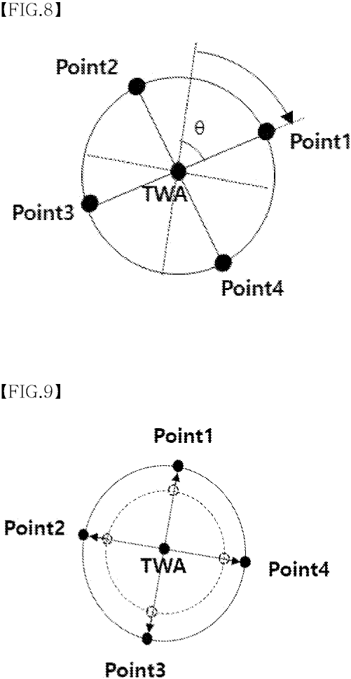

Referring to FIG. 8, FIG. 8 illustrates that a new peripheral position P is reset by rotating by an angle θ from the peripheral position P of FIG. 4. In order to generate a plurality of pieces of learning data by generating a new artificial image (not illustrated) different from the previously generated artificial image I, a new peripheral position P may be set by rotating a predetermined angle. Point 1, Point 2, Point 3, and Point 4, which are a plurality of peripheral positions P, may be rotated an angle θ to generate the blood flow data, the blood flow image, and the peripheral image 7000 at a new peripheral position P, and generate a new artificial image according to the central image 8000 and the new peripheral image (not illustrated). In addition, by changing the arrangement order of a new peripheral image and the central image 8000, it is possible to generate a plurality of new artificial images and generate a large amount of learning data.

FIG. 9 illustrates that a new peripheral position P is reset by changing a radius from the peripheral position P of FIG. 4. A new peripheral position P may be set by resetting the size of the radius different from the size of the radius from the center position T to the peripheral position P. A new peripheral position P may be reset by resetting the peripheral position P to a size of a radius smaller than the size of the set radius, but a new peripheral position P may also be reset by resetting the peripheral position P to a size of a radius larger than the size of the set radius. In addition to the size of the aneurysm and the degree of complexity of the shape of the aneurysm, it is possible to reset the size of the radius to set a new peripheral position P, and to generate a new artificial image accordingly to generate new learning data. In addition, new data may be created by changing the arrangement order of a new peripheral image and the central image 8000 according to the size of the radius.

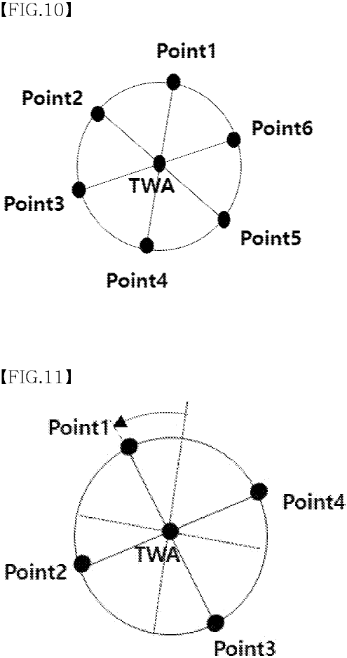

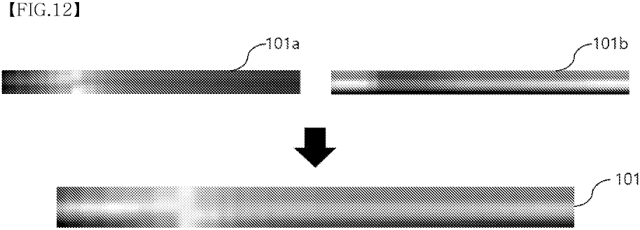

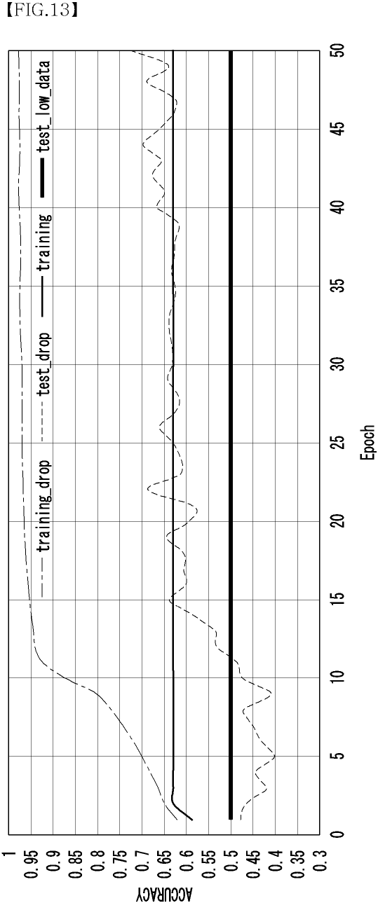

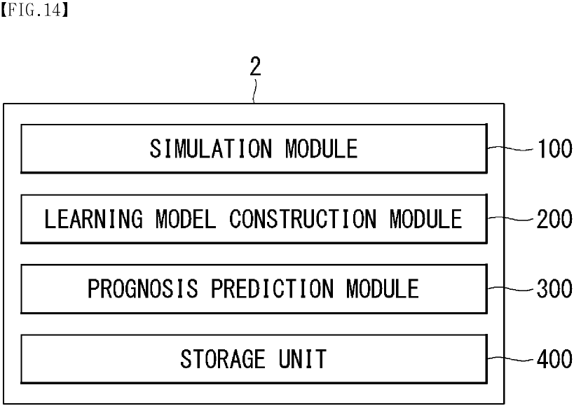

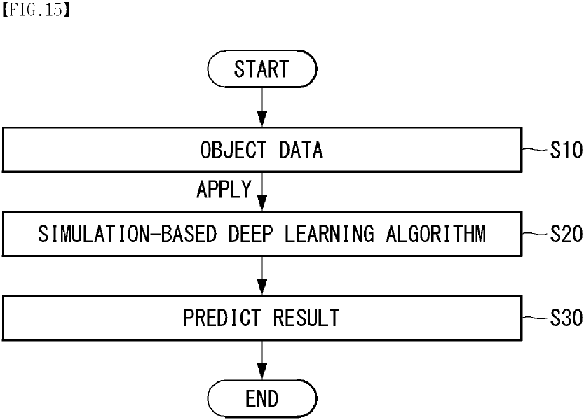

FIG. 10 illustrates that the number of peripheral positions P is changed. By increasing or decreasing the number of peripheral positions P, a new peripheral position P different from the peripheral position P of FIG. 4 may be generated, and a new artificial image may be generated accordingly. As illustrated in FIG. 10, a new peripheral position P may be set at six positions having a preset radius from the center position T. As illustrated in FIG. 10, six new peripheral positions P may be included, and a new peripheral image may be generated at each position to generate a new artificial image according to the central image 8000 and the new peripheral image. In addition, by changing the arrangement order of a new peripheral image and the central image 8000 according to the number of peripheral positions P, it is possible to generate different artificial images I.

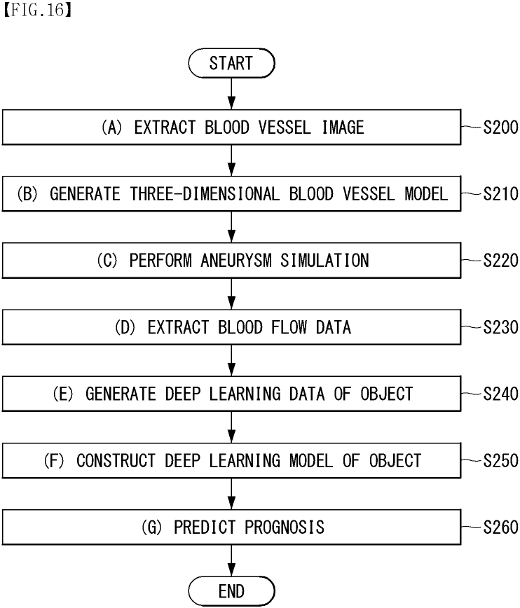

Referring to FIG. 11, FIG. 11 illustrates that a new peripheral position P is reset by rotating in the opposite direction to the direction in which FIG. 8 rotates θ. As in FIG. 8, the rotation angle may be changed, but a new peripheral position P may be set by changing the rotation direction. Also, by changing the angle in the opposite direction to reset the peripheral position P, a new peripheral position P may be set to generate a new peripheral image. Various artificial images I may be generated by regularly arranging the central image 8000 and new peripheral images or changing the arrangement order.

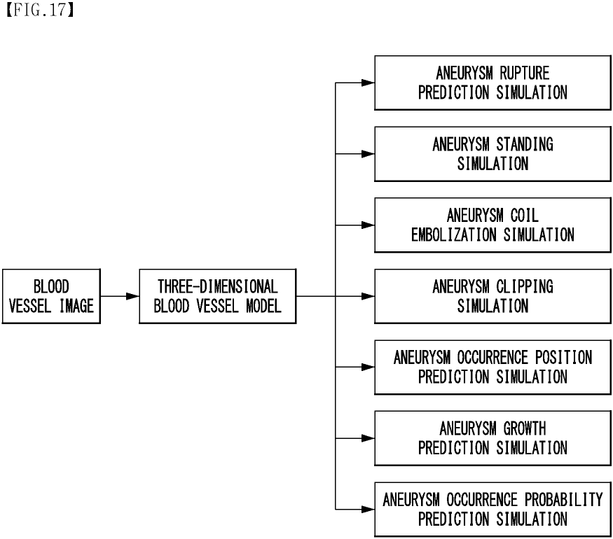

Referring to FIG. 12, a new artificial image NI may be generated by combining artificial images I including different blood flow data such as blood flow velocity, pressure, a strain rate, a deformation amount, the stress, force, WSS, and an OSI. For example, a new artificial image may be generated by merging an artificial image NIa for velocity and an artificial image NIb for pressure into one image. The new artificial image is formed by combining the artificial images I generated from different blood flow data, and has a feature that may consider various result values calculated from simulations at the same time. The new artificial image is not limited to the drawing illustrated in FIG. 12, and artificial images I including various pieces of blood flow data such as the strain rate, the deformation amount, the stress, the force, the WSS, and the OSI in addition to the blood flow velocity and the pressure may be formed by combining with each other.

Hereinafter, the deep learning result to which the method of augmenting aneurysm learning data of the present invention is applied will be described in detail with reference to FIG. 13.

FIG. 13 is a graph showing a deep learning result to which the method of augmenting aneurysm learning data of the present invention is applied.

Referring to FIG. 13, a difference in learning results is shown between a case where a simple simulation result is learned and a case where the method of augmenting aneurysm learning data of the present invention is applied and learned. When the simple simulation results are learned, a graph in which learning is not performed normally due to an insufficient amount of learning data may be viewed, and a graph in which accuracy is improved as a result of applying the method of augmenting aneurysm learning data of the present invention may be viewed. As described above, according to the present invention, it is possible to generate a large amount of learning data through the method of augmenting aneurysm learning data, thereby overcoming the limitation of the deep learning algorithm that requires a large amount of learning data, and improving accuracy.

Hereinafter, an aneurysm prediction system using a deep learning algorithm and an aneurysm prediction method using a deep learning algorithm will be described in detail with reference to FIGS. 14 to 20.

The aneurysm prediction system using the deep learning algorithm will be schematically described with reference to FIG. 14, the aneurysm prediction method using the deep learning algorithm will be described with reference to FIG. 15, and then the simulation-based deep learning algorithm will be described in detail with reference to FIGS. 16 to 20.

FIG. 14 is a configuration diagram of the aneurysm prediction system using the deep learning algorithm.

Referring to FIG. 14, an aneurysm prediction system 2 using a deep learning algorithm is a system in which a learning data module is learned based on hemodynamic data calculated through a simulation module 100 to accurately predict an aneurysm within a short time. In the aneurysm prediction system 2 using a deep learning algorithm, the simulation module 100 performs various simulations from data on one object, and the learning data module and a learning model construction module 200 may include a large amount of learning data and a learning model. The aneurysm prediction system 2 using a deep learning algorithm may include the simulation module 100, the learning model construction module 200, a prognosis prediction module 300, and a storage unit 400.

The simulation module 100 is for performing an aneurysm simulation with a blood vessel model of an object to form a deep learning algorithm. With one blood vessel model of an object, simulations, such as aneurysm rupture prediction, aneurysm stenting, aneurysm coil embolization, aneurysm clipping, aneurysm occurrence position prediction, aneurysm growth prediction, and aneurysm occurrence probability, are performed, and various pieces of blood flow data for each simulation are extracted and stored in the storage unit 400.

The learning model construction module 200 is for constructing an object deep learning model learned based on blood flow data calculated through the simulation module 100, and may form a simulation-based deep learning algorithm by learning the object deep learning data based on the blood flow data extracted through each aneurysm simulation. A plurality of object deep learning models constructed from the learning model construction module 200 may be stored in the storage unit 400.

The simulation module 100 and the learning model construction module 200 function as a central processing unit, the type of CPU may include a microprocessor, and the microprocessor may include a processing device in which an arithmetic logic operator, a register, a program counter, an instruction decoder, a control circuit, etc., are provided on at least one silicon chip.

In addition, the microprocessor may include a GPU for graphic processing of an image or video. The microprocessor may be implemented in the form of an SoC including a core and a GPU. The microprocessor may include a single core, a dual core, a triple core, a quad core, or a multiple-number core thereof.

In addition, the simulation module 100 and the learning model construction module 200 may include a graphic processing board including a GPU, a RAM, or a ROM on a separate circuit board electrically connected to the microprocessor.

The prognosis prediction module 300 predicts the aneurysm of the object in the object deep learning model included in the learning model construction module 200. The prognosis prediction module 300 may predict a blood vessel shape of an object and treatment methods such as coil embolization, clipping, and stenting according to the blood vessel shape, based on the object deep learning model.

The storage unit 400 may store the blood flow data, the object deep learning data, the object deep learning model, etc., extracted from the simulation module 100 and the learning model construction module 200, or store the pre-trained deep learning data, etc., and store variables, preset values, and the like used to execute the algorithm. The memory may include a non-volatile memory such as a ROM, a high-speed RAM, a magnetic disk storage device, a flash memory device, or other non-volatile semiconductor memory device. For example, the memory is a semiconductor memory device, and an SD memory card, an SDHC memory card, a mini SD memory card, a mini SDHC memory card, a TF memory card, a micro SD memory card, a micro SDHC memory card, a memory stick, a CF, an MMC, MMC micro, an XD card, etc., may be used. In addition, the memory may also include a network attached storage device that is accessed via a network.

Also, the storage unit 400 may include a CPU, a GPU, and various types of storage devices that are implemented with a microprocessor, etc. These devices may be provided on an embedded PCB.

FIG. 15 is a flowchart of an aneurysm prediction method using a deep learning algorithm.

Referring to FIG. 15, the aneurysm prediction method using a deep learning algorithm is for predicting the prognosis of an aneurysm of an object, and may predict an aneurysm by applying object data for predicting the aneurysm to the simulation-based deep learning algorithm. In other words, the aneurysm prediction system using a deep learning algorithm may predict a blood vessel shape of an object and the appropriate treatment method according to the blood vessel shape by applying the object data to the simulation-based deep learning algorithm. The aneurysm prediction system using a deep learning algorithm may load previously learned deep learning algorithm into the equipment to immediately predict the aneurysm of the object without requiring a separate learning process, generate the simulation deep learning algorithm in advance by learning the deep learning model with the hemodynamic factors calculated through the simulation inside the equipment, and then apply the object data to the deep learning algorithm when predicting the aneurysm to immediately predict a blood vessel shape of an object, an aneurysm, and a treatment method according to the aneurysm. The aneurysm prediction method is learned based on hemodynamic data calculated through hemodynamic simulation, so it is possible to accurately predict the aneurysm and minimize the time to predict the result. In particular, the aneurysm prediction system using a deep learning algorithm may form various learning models through different blood flow data and different simulations from data on one object, and construct a large number of deep learning models to predict various results. A simulation-based deep learning algorithm will be described in detail with reference to FIG. 16.

Hereinafter, a simulation-based deep learning algorithm for applying object data will be described in detail with reference to FIGS. 16 to 20.

Referring to FIG. 16, the simulation-based deep learning algorithm performs (a) extracting a blood vessel image (S200), (b) generating a three-dimensional blood vessel model (S210), (c) performing an aneurysm simulation (S220), (d) extracting blood flow data (S230), (e) generating deep learning data (S240), (f) constructing a deep learning model (S250), and (g) predicting a prognosis of an object (S260).

In order to form the simulation-based deep learning algorithm, a blood vessel image including a blood vessel shape of an object is extracted (S200). The blood vessel image of the object may include a three-dimensional blood vessel shape photographed from equipment such as digital subtraction angiography (DSA), magnetic resonance angiography (MRA), and computed tomography angiography (CTA) devices. A three-dimensional blood vessel model is generated from the blood vessel image of the object (S210). The three-dimensional blood vessel model is a blood vessel shape of an object formed in three dimensions from a blood vessel image, and after the three-dimensional blood vessel model of the object is generated, the aneurysm simulation may be performed with the three-dimensional blood vessel model (S220). More specifically described with reference to FIG. 17, the simulation-based deep learning algorithm may perform various aneurysm simulations with the three-dimensional blood vessel model. The aneurysm simulation may include an aneurysm rupture prediction simulation, an aneurysm stenting simulation, an aneurysm coil embolization simulation, an aneurysm clipping simulation, an aneurysm occurrence position prediction simulation, an aneurysm growth prediction simulation, an aneurysm occurrence probability prediction simulation, etc. Each of the various aneurysm simulations described above may be performed with one three-dimensional blood vessel model of an object. The aneurysm simulation in the present specification is described by taking the above-described simulation as an example, but is not limited thereto, and may include various simulations capable of predicting an aneurysm.

In the operation of performing the aneurysm simulation (S220), the aneurysm simulations such as rupture prediction, stenting, coil embolization, clipping, occurrence position prediction, growth prediction, and occurrence probability prediction may each be performed with the 3D blood vessel model to extract the blood flow data from each aneurysm simulation (S230). In addition, the operation of performing the aneurysm simulation (S220) is not limited to performing one aneurysm simulation, and a simulation may be performed by merging two or more of a plurality of aneurysm simulations, and other pieces of blood flow data may be extracted from the simulation (S230).

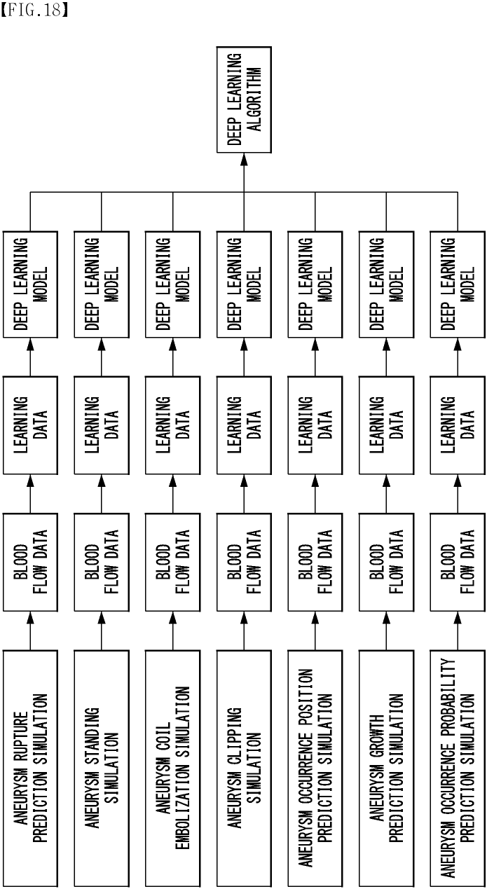

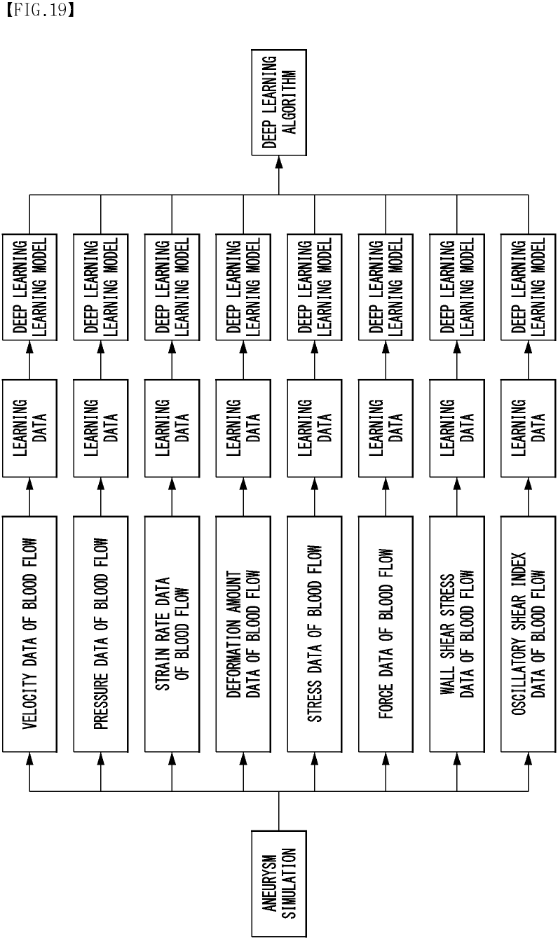

Referring to FIG. 18, a plurality of pieces of blood flow data may be extracted from simulation results for each aneurysm simulation by performing different aneurysm simulations from one three-dimensional blood vessel model of an object or performing the merged aneurysm simulation. By generating each deep learning data therefrom (S240), it is possible to construct the object deep learning model with the deep learning data (S250).

By performing a plurality of different aneurysm simulations from one 3D blood vessel model and extracting blood flow data for each aneurysm simulation to generate object learning data, a large number of deep learning models may be constructed. The object deep learning model may be constructed from the object deep learning data, but may also be constructed from the pre-trained deep learning data.

Meanwhile, the blood flow data may include at least one of the blood flow velocity, the pressure, the strain rate, the deformation amount, the stress, the force, the WSS, and the OSI.

Referring to FIG. 19, the deep learning algorithm may construct the object deep learning model by performing different simulations with the three-dimensional blood vessel model, but it is possible to build the object deep learning model according to one piece of blood flow data from each simulation. In other words, a plurality of pieces of blood flow data may be extracted from one aneurysm simulation, and a plurality of deep learning models may be constructed by generating the deep learning data according to each piece of blood flow data.

By applying different aneurysm simulations and blood flow data, and repeatedly performing operations (a) to (f), the simulation-based deep learning algorithm may be formed to construct a large number of deep learning models.

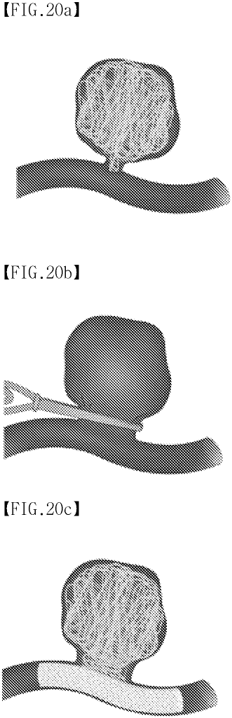

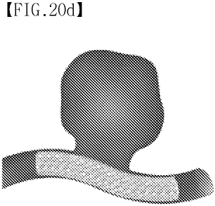

The prognosis of the object may be predicted by applying the object data for predicting an aneurysm to the simulation-based deep learning algorithm formed in this way (S260). The prognosis of the object may be predicted based on the deep learning model of the object, and as in FIGS. 20A, 20B, and 20C, the blood vessel shape of an object and appropriate treatment methods such as coil embolization, clipping, and stenting according to the blood vessel shape may be predicted. Also, as illustrated in FIG. 20D, the treatment method of coil embolization and stenting may be predicted to be applied at the same time, but the present invention is not limited thereto, and two or more types of different treatment methods may be predicted.

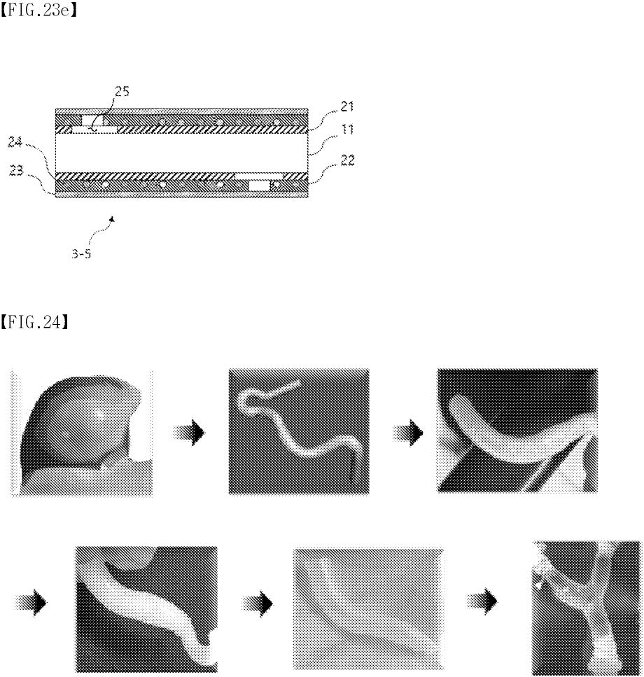

Hereinafter, a blood vessel for practice and a method of manufacturing a blood vessel for practice will be described in detail with reference to FIGS. 21 to 26. After describing the blood vessel for practice with reference to FIGS. 21 to 23, a method of manufacturing the blood vessel for practice will be described in detail with reference to FIGS. 24 to 26.

A blood vessel for practice will be described with reference to FIGS. 21 to 23.

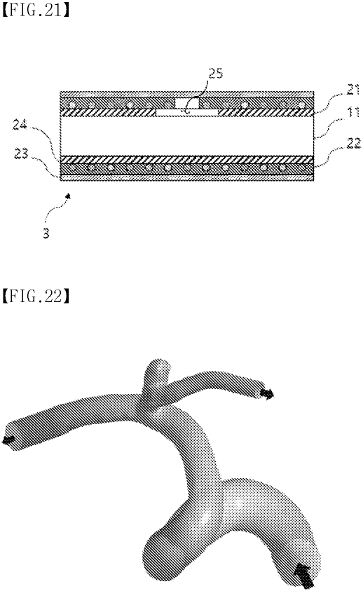

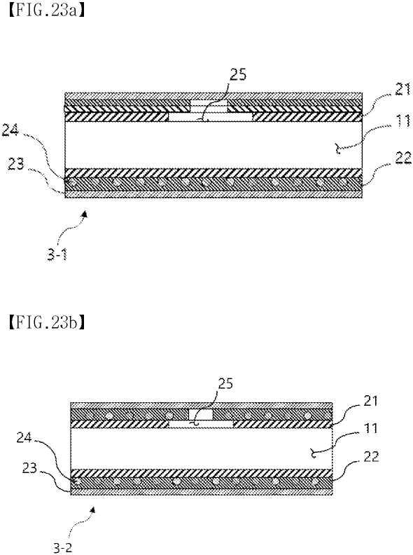

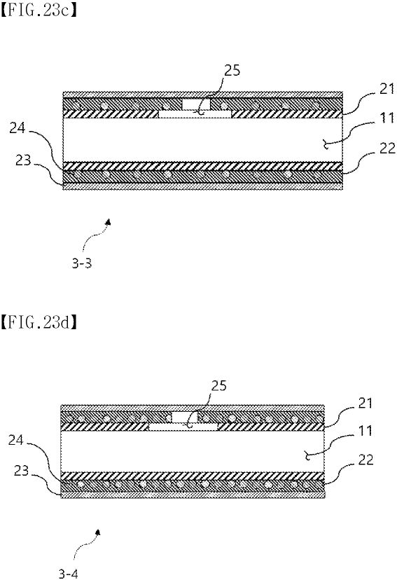

FIG. 21 is a cross-sectional view of a blood vessel for practice, FIG. 22 is a diagram illustrating a blood vessel model, and FIGS. 23 is a cross-sectional view of a blood vessel for practice illustrating a modified example of an auxiliary outer layer and an aneurysm inducing layer.

A blood vessel 3 for practice is a structure formed to increase the proficiency of medical staffs for whom it is difficult to access a procedure of vascular diseases related to an aneurysm and to secure stability of the procedure, and is formed similarly to reality based on the blood vessel shape and blood flow information of an object in which an aneurysm has occurred. The blood vessel 3 for practice is formed most similar to an actual blood vessel of each object having different blood vessels and blood flow information, and it is possible to increase the precision and proficiency of a procedure by providing a medical staff with a structure to reproduce the procedure before the procedure. Hereinafter, the blood vessel 3 for practice will be described in detail.

The blood vessel 3 for practice includes a first outer layer 21, a second outer layer 22, a third outer layer 23, and an aneurysm inducing layer 25 to form a structure similar to an actual blood vessel. Specifically, the blood vessel 3 for practice includes the first outer layer 21 that is formed by being applied on an outer circumferential surface of a blood vessel core 1000 output in three dimensions according to blood vessel data and blood flow data including a blood vessel shape and blood flow analysis information and includes an inner space 11 formed by removing the blood vessel core 1000, the second outer layer 22 that is formed by applying a component different from the first outer layer 21 according to the blood vessel data and the blood flow data to an outer circumferential surface of the first outer layer 21, the third outer layer 23 that is formed by applying a component different from the first outer layer 21 and the second outer layer 22 according to the blood vessel data and the blood flow data to an outer circumferential surface of the second outer layer 22, and an aneurysm inducing layer 25 that is recessed by removing at least a portion of the first outer layer 21, the second outer layer 22, and the third outer layer 23 from the inner space 11.

Prior to the description of each configuration of the blood vessel 3 for practice, the blood vessel data refers to a three-dimensional blood vessel shape of an object extracted by angiography, a thickness of the blood vessel of the object, components constituting the blood vessel, information on physical properties such as density, and the like, and the blood flow data refers to data common to each object, ancillary tissue surrounding a blood vessel or near the blood vessel, and the like. The three-dimensional blood vessel model refers to a blood vessel shape of an object formed in three dimensions according to blood vessel data and blood flow data, and the blood vessel core 1000 refers to a three-dimensional structure by which a three-dimensional blood vessel model extracted from the blood vessel data and the blood flow data is output using a 3D printer. The blood vessel data and the blood flow data will be described later in detail through the method of manufacturing blood vessels for practice.

The first outer layer 21 is a coating layer applied on an outer surface of the blood vessel core 1000 output based on the three-dimensional blood vessel model of the object, and is made of a material according to the analysis of the blood vessel data and the blood flow data. The thickness and components of the first outer layer 21 may be calculated according to the blood vessel data and the blood flow data, and the first outer layer 21 may be manufactured by mixing, for example, at least one of hyaluronic acid, gelatin, glycerol, alginate, fibroblasts of a mixture of gelatin and alginate, endothelial cells, human umbilical vein endothelial cells (HUVECs), and fibrinogen, and may be applied on an outer surface of the blood vessel core 1000. After the first outer layer 21 is applied on an outer circumferential surface of the blood vessel core 1000 and cured, the first outer layer 21 is formed in a cylindrical shape in which the blood vessel core 1000 is separated from the inner circumferential surface to form an empty space therein. The first outer layer 21 may be formed in a serpentine shape as illustrated in FIG. 22 according to a three-dimensional blood vessel model of an object. The drawing illustrated in FIG. 21 is an enlarged view of a cut part of the blood vessel 3 for practice, and the blood vessel 3 for practice is not limited to the shape illustrated in FIG. 21 and may be formed in a serpentine shape, such as the shape illustrated in FIG. 22. The second outer layer 22, the third outer layer 23, an auxiliary outer layer 24, and the like may be applied on the outer surface of the first outer layer 21.

The second outer layer 22 and the third outer layer 23 are structures forming a part of the blood vessel 3 for practice. The second outer layer 22 is applied on the outer surface of the first outer layer 21 and the third outer layer 23 forms a coating layer applied on the outer surface of the second outer layer 22. Like the first outer layer 21, the second outer layer 22 and the third outer layer 23 are made of a thickness and components according to the analysis of the blood vessel data and the blood flow data, and are made of components in which, for example, at least one of hyaluronic acid, gelatin, glycerol, alginate, fibroblasts of a mixture of gelatin and alginate, endothelial cells, human umbilical vein endothelial cells (HUVEC), and fibrinogen, is mixed. The second outer layer 22 and the third outer layer 23 may be applied and formed in close contact with the surfaces of the first outer layer 21 and the second outer layer 22 to form a thin film.

The first outer layer 21, the second outer layer 22, and the third outer layer 23 form a structure 2000 made of different thicknesses and components based on the blood vessel data and the blood flow data, and may have the auxiliary outer layer 24 formed between at least one layer.

At least one auxiliary outer layer 24 is formed between the first outer layer 21, the second outer layer 22, and the third outer layer 23, and forms a part of the structure 2000 for controlling the strength of the blood vessel 3 for practice. The auxiliary outer layer 24 is made of a separate material having different strength from the first outer layer 21, the second outer layer 22, and the third outer layer 23. The auxiliary outer layer 24 may be closely disposed to surround the outer circumferential surface of the first outer layer 21 or the second outer layer 22, and as illustrated in the drawing, the case where the auxiliary outer layer 24 may be closely disposed to surround the outer circumferential surface of the first outer layer 21 will be described by way of example.

More specifically, referring to FIGS. 23, the auxiliary outer layer 24 may have various thicknesses and shapes. The auxiliary outer layer 24 may be formed on the outer circumferential surface of the first outer layer 21 to differently express the degree of deformation of the blood vessel when a force is applied to the blood vessel in a transverse or longitudinal direction. The auxiliary outer layer 24 may adjust the strength of the blood vessel differently by changing the shape formed in close contact with the outer circumferential surface of the first outer layer 21. The auxiliary outer layer 24 may be formed in the same shape as a thin and slender cylindrical shim, or may be formed as a thin film. The case where the auxiliary outer layer 24 is formed in an elongated cylindrical shape as illustrated in FIG. 21 and is disposed to surround the outer circumferential surface of the first outer layer 21 along the longitudinal direction of the structure 2000 will be described by way of example. However, the auxiliary outer layer 24 is formed in the form of a thin film to form the coating layer applied on the outer circumferential surface of the first outer layer 21 as illustrated in FIG. 23A, and may be disposed between the first outer layer 21 and the second outer layer 22. In addition, auxiliary outer layers 24 are disposed to surround the outer circumferential surface of the first outer layer 21 in an oblique line along the longitudinal direction of the structure 2000, and as illustrated in FIGS. 23B to 23D, the plurality of outer layers 24 disposed up and down with respect to the inner space 11 may face each other to form a symmetry or may be disposed to be shifted from each other, and may be disposed to narrow or widen the spaced interval along the longitudinal direction of the structure 2000. As the auxiliary outer layer 24 may be formed in various shapes, the strength of the blood vessel core 1000 for practice may be adjusted differently for each position, and the degree of deformation of the blood vessel may be expressed differently so that characteristics of the directionality of a blood vessel of an object may be formed to be similar to the actual characteristics. In addition, the auxiliary outer layer 24 may be made of a material having different thicknesses or strength depending on a position of a circumferential surface of a cross-section perpendicular to the longitudinal direction, and may be formed by mixing a membrane and a cylindrical shape.

In the structure 2000 including the first outer layer 21, the second outer layer 22, and the third outer layer 23, the aneurysm inducing layer 25 cut from the inner space 11 is formed.

The aneurysm inducing layer 25 is an empty space recessed from the inner circumferential surface of the structure 2000, and may be formed by cutting at least a portion of the inside of the structure 2000. The aneurysm inducing layer 25 may be formed by cutting at least one of the first outer layer 21, the second outer layer 22, the third outer layer 23, and the auxiliary outer layer 24 at the position where an actual aneurysm occurs in the blood vessel of the object, and as illustrated in the drawings, may be formed by cutting the first outer layer 21 and the auxiliary outer layer 24. The aneurysm inducing layer 25 is formed at the position where an aneurysm occurs in the actual blood vessel of the object calculated from the blood vessel data and the blood flow data. In addition, as illustrated in FIG. 23E, the aneurysm inducing layer 25 may be formed at different positions for each object by calculating a position formed on the inner circumferential surface of the structure 2000 based on the blood vessel data and the blood flow data. The aneurysm inducing layer 25 may swell as a pump 26 is connected to both end portions of the blood vessel 3 for practice to make a fluid 27 flow into the inner space 11. Accordingly, the aneurysm inducing layer 25 may form an aneurysm similar to the actual blood vessel of the object in the blood vessel 3 for practice, establish a highly stabile procedure plan by increasing the surgical application ability of medical staff, and form the blood vessel 3 for practice of each object the most similar to the actual blood vessel.

Meanwhile, the case where the aneurysm inducing layer 25 of the blood vessel 3 for practice according to an embodiment is formed by removing at least a portion of the first outer layer 21, the second outer layer 22, and the third outer layer 23 from the inner space 11 will be described by way of example. However, the present invention is not limited thereto, and the aneurysm inducing layer 25 may be formed by radiating a portion of the structure 2000 with heat or UV light, or may be formed by applying a portion having different strength to a portion of the structure 2000. A blood vessel 3a for practice according to another embodiment will be described below in detail with reference to FIGS. 27 and 28.

Hereinafter, a method of manufacturing a blood vessel for practice will be described in detail with reference to FIGS. 24 to 26.

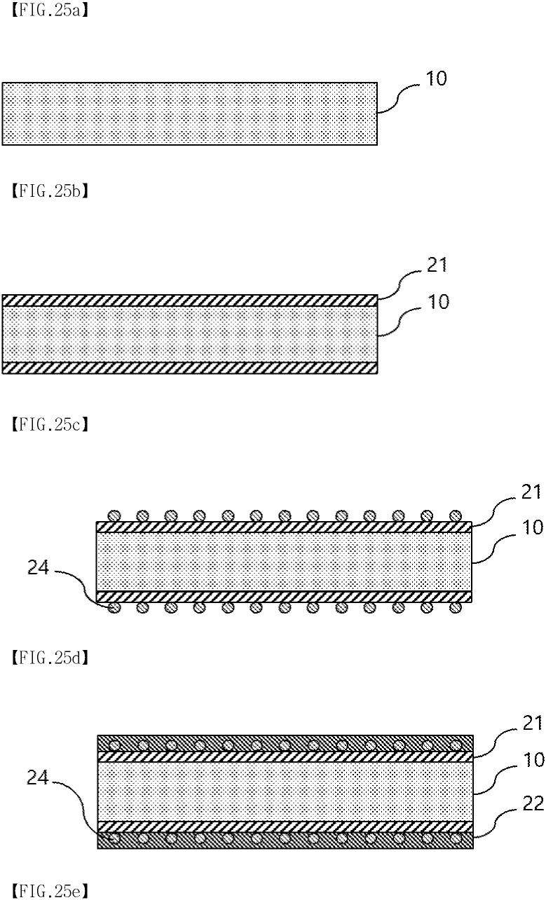

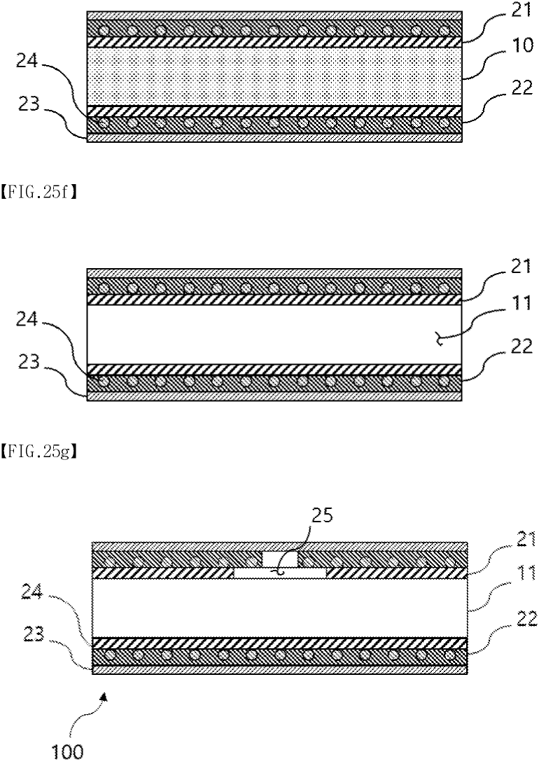

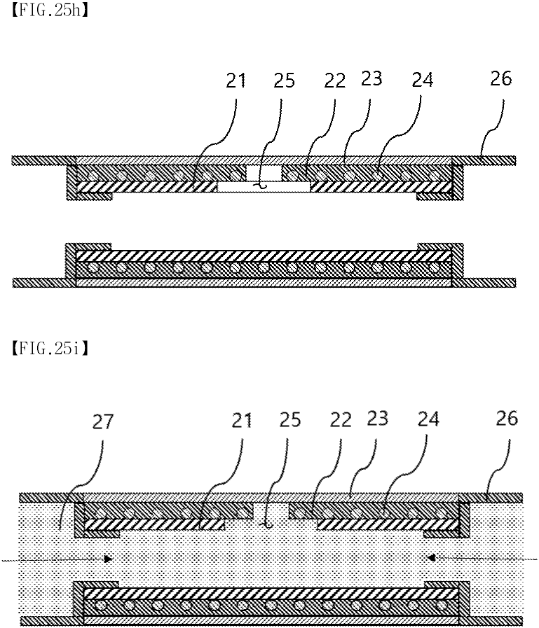

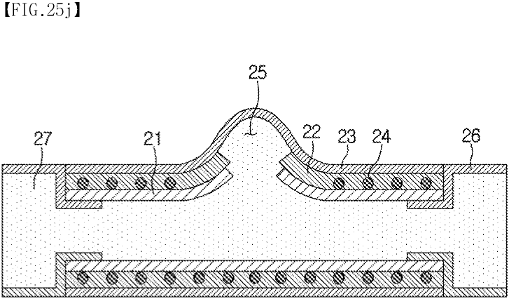

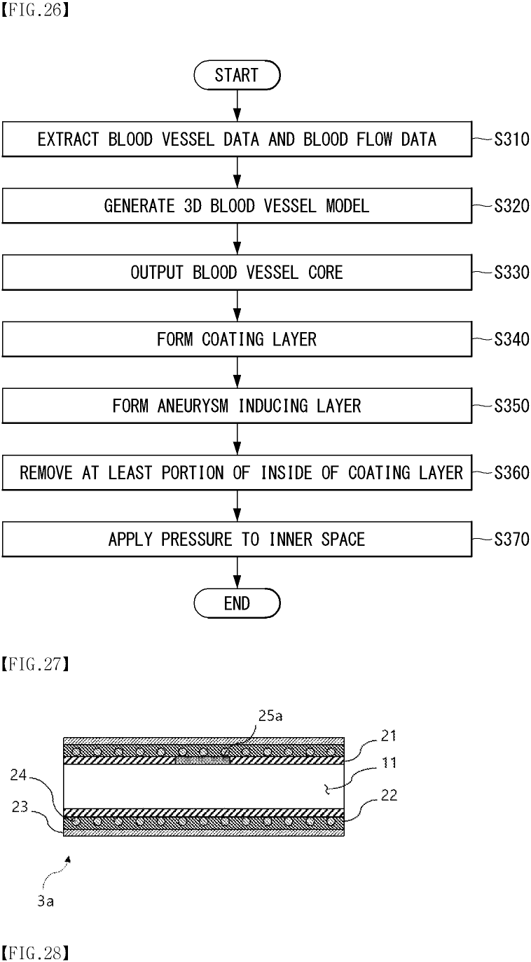

FIG. 24 is a photograph showing a manufacturing procedure of a blood vessel for practice, FIGS. 25 is a diagram illustrating a method of manufacturing a blood vessel for practice, and FIG. 26 is a flowchart of the method of manufacturing a blood vessel for practice.

The method of manufacturing a blood vessel for practice will be briefly described with reference to a photo of manufacturing the blood vessel for practice step by step in FIG. 24, which will be described below in detail with reference to FIGS. 25 and 26.

The method of manufacturing a blood vessel for practice includes extracting blood vessel data and blood flow data including a blood vessel shape and blood flow analysis information of an object (S300), forming a three-dimensional blood vessel model according to the blood vessel data and the blood flow data (S310), outputting a blood vessel core 1000 based on the blood vessel model (S320), forming a structure 2000 by applying a first outer layer 21, a second outer layer 22, and a third outer layer 23, which are different from each other, according to the blood vessel data and the blood flow data to an outer surface of the blood vessel core 1000, removing the blood vessel core 1000 in the structure 2000 (S340), forming an aneurysm inducing layer 25 by removing at least a portion of the inside of the structure 2000 from an inner space 11 from which the blood vessel core 1000 is removed (S350, see FIG. 25G), and applying pressure to the inner space 11 by connecting a pump 26 to both open end portions of the structure 2000 (S360).

The method of manufacturing a blood vessel for practice extracts the blood vessel data and the blood flow data including the blood vessel shape and blood flow analysis information of the object, and predicts changes in thickness and physical properties of the blood vessel. The 3D blood vessel model is generated based on the blood vessel data and the blood flow data, and the blood vessel core 1000 is output using a 3D printer based on the blood vessel model stored in a 3D file. The structure 2000 is formed by applying a coating layer to the outer surface of the blood vessel core 1000 output by the 3D printer multiple times, curing the coating layer, and then removing the blood vessel core therein. Thereafter, the pump 26 may be connected to the structure 2000 to supply a fluid 27, thereby forming the blood vessel 3 for practice similar to the actual blood vessel model.

Referring to FIGS. 25 and 26, the method of manufacturing a blood vessel for practice is described in more detail. In the operation of extracting the blood vessel data and the blood flow data (S300), the blood vessel shape and the blood flow analysis information of the object are calculated. The blood vessel data includes a three-dimensional blood vessel shape of the object extracted by angiography, the thickness of the blood vessel of the object, the components constituting the blood vessel, and information on physical properties such as density and strength, and the like. The blood flow data may include not only the blood flow information data of each object, but also ancillary tissues surrounding the blood vessel or near the blood vessel, for example, incidental tissues inside a skull when the skull is opened, environmental factors, and the like.

After the blood vessel data and the blood flow data are extracted, the three-dimensional blood vessel model according to the blood vessel data and the blood flow data is generated (S310). The three-dimensional blood vessel model refers to a blood vessel shape of an object formed in three dimensions according to the blood vessel data and the blood flow data, and may include, for example, 3D modeling of a blood vessel shape of an object generated by angiography or the like.

After generating a 3D blood vessel model most similar to the actual blood vessel of the object, the blood vessel core 1000 based on the blood vessel model is output (S320). FIG. 25A illustrates a cross-sectional view of a portion of the blood vessel core 1000, and the blood vessel core 1000 is output based on the blood vessel model. The blood vessel core 1000 in FIG. 25A is a diagram illustrating a portion of the whole output based on the overall blood vessel model in FIG. 22 described above, and the blood vessel core 1000 of the blood vessel 3 for practice formed based on the actual blood vessel may be formed in a serpentine shape.

After outputting the blood vessel core 1000, the structure 2000 is formed by applying the first outer layer 21, the second outer layer 22, and the third outer layer 23, which are different from each other, according to the blood vessel data and the blood flow data to the outer surface of the blood vessel core 1000 (S330). Referring to FIGS. 25B to 25E, the drawings for forming the structure 2000 by applying the coating layers of the first outer layer 21, the second outer layer 22 and the third outer layer 23 to the outer circumferential surface of the blood vessel core 1000 are illustrated step by step. The first outer layer 21 is formed by applying components prepared by mixing at least one of hyaluronic acid, gelatin, glycerol, alginate, fibroblasts of a mixture of gelatin and alginate, endothelial cells, human umbilical vein endothelial cells (HUVECs), and fibrinogen according to the blood vessel data and the blood flow data to the outer surface of the blood vessel core 1000. An auxiliary outer layer 24 having an elongated shim shape is closely disposed on the outer surface of the first outer layer 21 to surround the first outer layer 21 along the longitudinal direction, and the second outer layer 22 is applied on the cured outer surfaces of the first outer layer 21 and the auxiliary outer layer 24. To continue, the third outer layer 23 is applied on the outer surface of the second outer layer 22. The first outer layer 21, the auxiliary outer layer 24, the second outer layer 22, and the third outer layer 23 constituting the structure 2000 may have different components prepared by mixing at least one of hyaluronic acid, gelatin, glycerol, alginate, fibroblasts of a mixture of gelatin and alginate, endothelial cells, human umbilical vein endothelial cells (HUVECs), and fibrinogen based on the blood vessel data and the blood flow data, and different thicknesses.

After all the first outer layer 21, the auxiliary outer layer 24, the second outer layer 22, and the third outer layer 23 applied on the outer circumferential surface of the blood vessel core 1000 are cured to form the structure 2000, the blood vessel core 1000 inside the structure 2000 is removed (S340). FIG. 25F illustrates the structure 2000 from which the blood vessel core 1000 is removed, and as illustrated in FIG. 25F, the blood vessel core 1000 disposed inside the first outer layer 21 is removed therein, and the inner space 11 is formed inside the structure 2000. In the structure 2000, an empty space from which the blood vessel core 1000 is removed is formed therein, and the first outer layer 21, the auxiliary outer layer 24, the second outer layer 22, and the third outer layer 23 are sequentially stacked and formed in a cylindrical shape.

In the structure 2000 thus formed, the aneurysm inducing layer 25 is formed by removing at least a portion of the inside of the structure 2000 from the inner space 11 (S350). FIG. 25G illustrates that the aneurysm inducing layer 25, which is a space recessed from the inner circumferential surface of the structure 2000 by cutting a portion of the inside of the structure 2000, is formed. The aneurysm inducing layer 25 may be formed according to the thickness and position calculated according to the blood vessel data and the blood flow data as well as the first outer layer 21 and the auxiliary outer layer 24. For example, the aneurysm inducing layer 25 is formed at the position where an aneurysm occurs in the actual blood vessel of the object calculated from the blood vessel data and the blood flow data. In addition, the aneurysm inducing layer 25 may be formed at different positions for each object by calculating the position formed on the inner circumferential surface of the structure 2000 based on the blood vessel data and the blood flow data.

In the structure 2000 in which the aneurysm inducing layer 25 is formed, the pump 26 is connected to both open end portions so that pressure is applied to the inner space 11 (S360). As illustrated in FIGS. 25H and 25I, when the pump 26 is connected to both open end portions of the blood vessel 3 for practice to supply the fluid 27 to the inner space 11, the blood vessel 3 for practice similar to the actual blood vessel of the object may be formed. When the fluid 27 is continuously supplied to the inner space 11 of the structure 2000, as illustrated in FIG. 25J, the aneurysm inducing layer 25 swells, so that a blood vessel in which an aneurysm actually occurs may be implemented. In this way, the blood vessel 3 for practice manufactured by the method of manufacturing a blood vessel for practice is formed, and the aneurysm inducing layer 25 of the blood vessel 3 for practice may form an aneurysm similar to the actual blood vessel of the object, thereby increasing the surgical application ability of medical staff and establishing a highly stable procedure plan. In addition, the blood vessel 3 for practice is formed based on the blood vessel data and the blood flow data to form an aneurysm at the same position as the actual blood vessel, and the same thickness, touch, and the like as the actual blood vessel may be implemented, thereby enabling more precise procedure practice for medical staff.

Hereinafter, a blood vessel for practice and a method of manufacturing the blood vessel for practice according to another embodiment will be described in detail with reference to FIGS. 27 to 30.

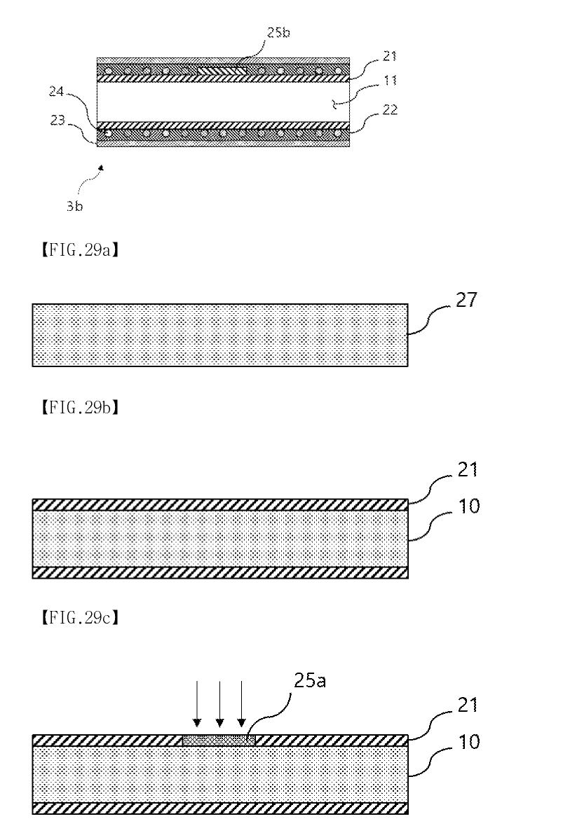

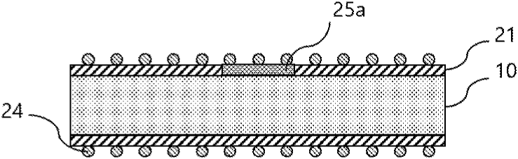

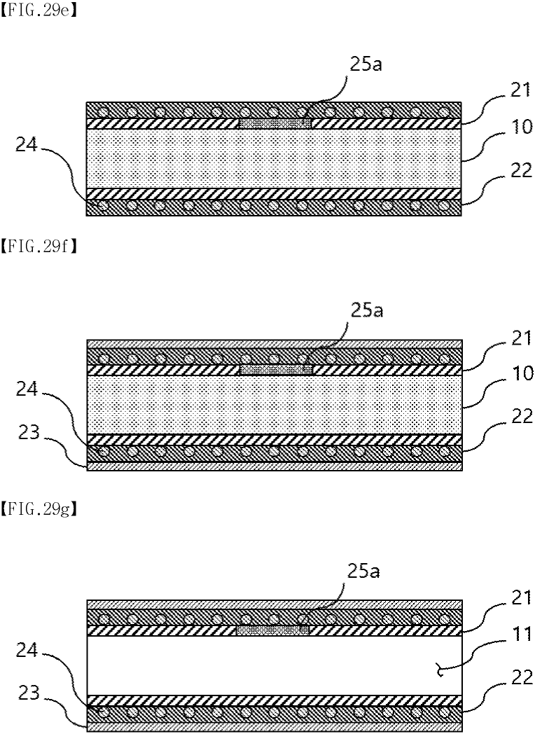

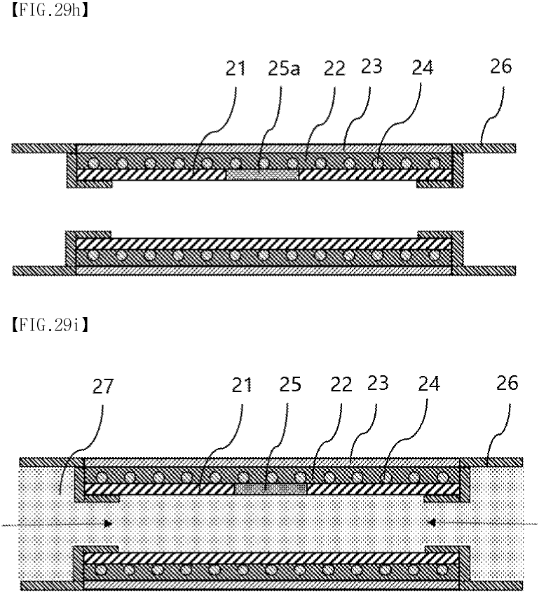

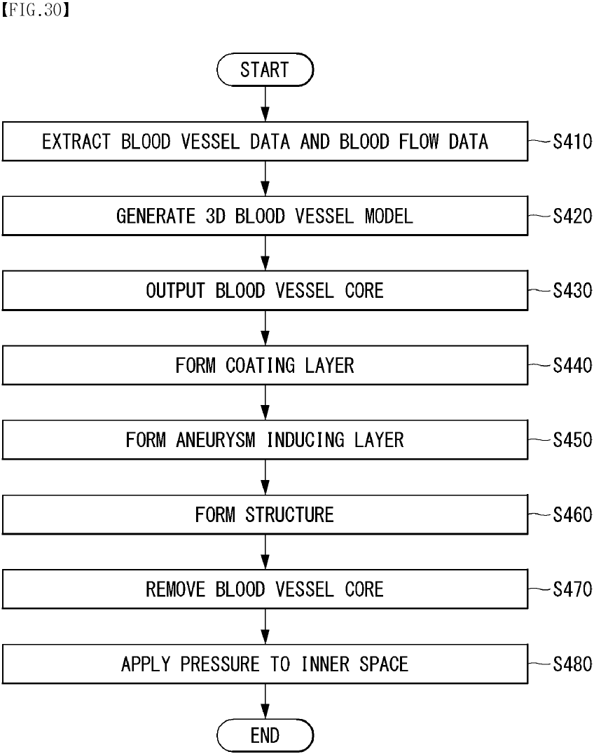

FIGS. 27 and 28 are diagrams of a blood vessel for practice according to another embodiment, FIGS. 29 is a diagram illustrating a method of manufacturing a blood vessel for practice according to another embodiment, and FIG. 30 is a flowchart of a method of manufacturing a blood vessel for practice according to another embodiment.

A blood vessel 3a for practice according to another embodiment is substantially the same as the above-described embodiment except for an aneurysm inducing layer 25a. Accordingly, the same reference numerals are assigned to the same components as those already described, and detailed descriptions thereof will be omitted.

The blood vessel 3a for practice according to another embodiment includes the aneurysm inducing layer 25a that is formed by radiating heat or UV light or applying components having different strength to an outer surface of at least one of a first outer layer 21, a second outer layer 22, and a third outer layer 23.

In one embodiment, the aneurysm inducing layer 25 may be formed by changing the thickness of the structure 2000 by removing the inner coating layer, but in another embodiment, the aneurysm inducing layer 25a may also be formed by changing the physical properties of the structure 2000. Unlike one embodiment in which the aneurysm inducing layer 25 is formed by removing at least a portion of the inside of the structure 2000, the aneurysm inducing layer 25a is formed by radiating heat or UV light or applying components having different strength to the outer surface of at least one of the first outer layer 21, the second outer layer 22, and the third outer layer 23.

Referring to FIG. 27, the aneurysm inducing layer 25a may be formed by radiating heat or UV light to a specific position of the outer surface of at least one of the first outer layer 21, the second outer layer 22, and the third outer layer 23. The aneurysm inducing layer 25a is formed by radiating heat or UV light to a specific position of the outer surface of the first outer layer 21, so that the strength thereof may vary from the remaining positions to which heat or UV light is not radiated. The second outer layer 22 may be applied on the entire outer surface of the first outer layer 21 whose specific position is irradiated with heat or UV light, and the heat or UV light may be radiated to the position of the second outer layer 22 in contact with the aneurysm inducing layer 25a formed in the first outer layer 21. In the same way, heat or UV light may be radiated to the third outer layer 23 facing the aneurysm inducing layer 25a formed in the first outer layer 21 and the second outer layer 22 to form the aneurysm inducing layer 25a. The aneurysm inducing layer 25a thus formed is formed to have different strength from the remaining portions where the aneurysm inducing layer 25a is not formed, and thus, when pressure is applied to an inner space 11, an aneurysm similar to an actual blood vessel may be formed. In the present specification, the case where heat or UV light is radiated to each outer layer of the structure 2000 to form the aneurysm inducing layer 25a is described by way of example, but the present invention is not limited thereto, and heat or UV light is radiated to the outer surface of at least one of the first outer layer 21, the second outer layer 22, and the third outer layer 23, or heat or UV light is radiated to the outer surface of the structure 2000 formed by coating the first outer layer 21, the second outer layer 22, and the third outer layer 23, so that the aneurysm inducing layer 25a may also be formed. Meanwhile, it is not limited to heat or UV light to adjust the strength of a portion of the aneurysm inducing layer 25a by changing the physical properties of the structure 2000.

Referring to FIG. 28, an aneurysm inducing layer 25b may be formed by applying components having different strength to the outer surface of at least one of the first outer layer 21, the second outer layer 22, and the third outer layer 23. When the first outer layer 21, the second outer layer 22, and the third outer layer 23 are sequentially applied on the outer surface of the blood vessel core 1000, the aneurysm inducing layer 25b may be formed by applying components having different strength to a portion of the outer surfaces of the first outer layer 21, the second outer layer 22, and the third outer layer 23. For example, the aneurysm inducing layer 25b may be formed by applying components having a weaker strength than that of the first outer layer 21 to the outer surface of the first outer layer 21, and the structure 2000 may be formed by applying the second outer layer 22 and the third outer layer 23 to the outer surface of the aneurysm inducing layer 25b. In this case, a step is formed while the aneurysm inducing layer 25b is applied on the outer surface of the first outer layer 21, but when the second outer layer 22 is applied on the outer surface of the aneurysm inducing layer 25b, the second outer layer 22 may be applied to have a small thickness at a specific position where the aneurysm inducing layer 25b is formed or may not be applied to only the specific position where the aneurysm inducing layer 25b is formed. In other words, the second outer layer 22 may be coplanar with the aneurysm inducing layer 25b to form a coating layer in which the position where the aneurysm inducing layer 25b is formed does not protrude. The specific position of the structure 2000 in which the aneurysm inducing layer 25b is formed is made of a component having a lower strength than that of each of the first outer layer 21, the second outer layer 22, and the third outer layer 23 and is disposed to be surrounded by the structure 2000. Accordingly, the aneurysm inducing layer 25b may be easily deformed while swelling by the pressure applied to the inner space 11 of the structure 2000. The aneurysm inducing layer 25b is not limited to the illustrated drawings, and may be applied to occupy a portion of the third outer layer 23 as well as the second outer layer 22. Meanwhile, the aneurysm inducing layer 25b may be formed opposite to the illustrated drawing, and the aneurysm inducing layer 25b may be applied on the remaining portion except for the position where the aneurysm inducing layer 25b is formed in FIGS. 29. For example, the aneurysm inducing layer 25b may be formed by various methods such as applying a component having high strength to the remaining outer surface except for a predetermined position on the outer surface of the first outer layer 21 and forming the aneurysm inducing layer 25b at a predetermined position that is not applied, etc. A blood vessel 3b for practice may be formed by applying the second outer layer 22 and the third outer layer 23 to the outer surface of the aneurysm inducing layer 25b and removing the blood vessel core 1000 inside the structure 2000.

Hereinafter, a method of manufacturing a blood vessel for practice according to another embodiment will be described in detail with reference to FIGS. 29 to 30.

FIGS. 29 is a diagram illustrating a method of manufacturing a blood vessel for practice, and FIG. 30 is a flowchart of a method of manufacturing a blood vessel for practice.

The method of manufacturing a blood vessel for practice includes extracting blood vessel data and blood flow data including blood vessel shape and blood flow analysis information of an object (S410), generating a three-dimensional blood vessel model according to the blood vessel data and the blood flow data (S420), outputting a blood vessel core 1000 based on the blood vessel model (S430), forming a coating layer by applying a first outer layer 21 according to the blood vessel data and the blood flow data to an outer surface of the blood vessel core 1000 (S440), forming an aneurysm inducing layer 25a on at least a portion of the outer surface of the first outer layer 21 (S450), forming a structure 2000 including the first outer layer 21, a second outer layer 22, and a third outer layer 23 by repeatedly applying a coating layer to the outer surface of the first outer layer 21 (S460), removing the blood vessel core 1000 inside the structure 2000 (S470), and applying pressure to an inner space 11 by connecting a pump 26 to both open end portions of the structure 2000 (S480).

The method of manufacturing a blood vessel for practice according to another embodiment is substantially the same as the method of manufacturing a blood vessel for practice according to one embodiment described above, except for forming the coating layer (S430), forming the aneurysm inducing layer 25a (S440), forming the structure 2000 (S450), and removing the blood vessel core 1000 (S460) of FIGS. 29B to 29G. Accordingly, the detailed description of the operation (S400) of extracting the blood vessel data and the blood flow data, the operation (S410) of generating the three-dimensional blood vessel model, the operation (S420) of outputting the blood vessel core 1000, and the operation (S470) of applying pressure to the inner space, which are already described manufacturing operations, and FIGS. 29A, 29H, and 298I including the same will be omitted.

In the method of manufacturing a blood vessel for practice according to another embodiment, the coating layer is formed by applying the first outer layer 21 to the outer circumferential surface of the blood vessel core 1000 output in three dimensions according to the blood flow data and the blood vessel data including the blood vessel shape and the blood flow analysis information (S430). Referring to FIG. 29B, the first outer layer 21 is formed by applying the components according to the blood vessel data and the blood flow data to the outer surface of the blood vessel core 1000 (S430). After the first outer layer 21 is applied and the applied first outer layer 21 is cured, the aneurysm inducing layer 25a is formed on at least a portion of the outer surface of the first outer layer 21 (S440). When forming the aneurysm inducing layer 25a on the outer surface of the first outer layer 21, the aneurysm inducing layer 25a may be formed by radiating heat or UV light or applying components having different strength to at least a portion of the outer surface of the first outer layer 21. For example, as illustrated in FIG. 29C, by radiating heat or UV light to a specific position of the first outer layer 21 where the aneurysm inducing layer 25a is to be formed to change the strength of a portion of the first outer layer 21, the aneurysm inducing layer 25a may be formed in a portion of the first outer layer 21. In addition, as in FIG. 28 described above, the aneurysm inducing layer 25a may be formed by applying a component different from the first outer layer 21 to the specific position of the first outer layer 21 where the aneurysm inducing layer 25a is to be formed. Referring to FIG. 29D, as in one embodiment, an auxiliary outer layer 24 may be formed on the outer surface of the first outer layer 21 on which the aneurysm inducing layer 25a is formed, and since the auxiliary outer layer 24 has the same configuration as in one embodiment and is formed on the surface of the first outer layer 21 in the same way, a detailed description thereof will be omitted. To continue, referring to FIGS. 29E and 29F, by repeatedly applying the coating layers of the second outer layer 22 and the third outer layer 23 to the outer surface of the first outer layer 21 on which the aneurysm inducing layer 25a and the auxiliary outer layer 24 are formed, the structure 2000 including the first outer layer 21, the second outer layer 22, and the third outer layer 23 is formed (S450). In this case, in the operation of forming the structure 2000 (S450), the second outer layer 22 and the third outer layer 23 may be continuously applied, but in addition to the drawings illustrated, after the second outer layer 22 is cured, heat or UV light may be radiated to the outer surface of the second outer layer 22 to form the aneurysm inducing layer 25a on the second outer layer 22, and the third outer layer 23 may be formed by being applied on the outer surface of the second outer layer 22 on which the aneurysm inducing layer 25a is formed. In this way, in the operation of forming the structure 2000 (S450), the operation (S440) of forming the aneurysm inducing layer 25a on the outer surface of at least one of the second outer layer 22 and the third outer layer 23 may be repeatedly performed. However, the present invention is not limited thereto, and the aneurysm inducing layer 25a may be formed on at least a portion of the first outer layer 21, the second outer layer 22 and the third outer layer 23 through a method other than the method of radiating heat or UV light.

In the structure 2000 that has undergone the operation of forming the coating layer (S430), the operation of forming the aneurysm inducing layer 25a (S440), and the operation of forming the structure 2000 (S450), as illustrated in FIG. 29G, the blood vessel core 1000 formed therein may be removed (S460). In the method of removing a blood vessel for practice according to another embodiment, the case in which after the structure 2000 including the aneurysm inducing layer 25a, the first outer layer 21, the second outer layer 22, and the third outer layer 23 is formed, the blood vessel core 1000 is removed to form the inner space 11 is described by way of example, but the operation of removing the blood vessel core 1000 (S460) may be applied after the operation (S430) in which the first outer layer 21 is applied to form the coating layer.

In the blood vessel 3 for practice, the aneurysm inducing layer 25 may be formed by removing a portion of the plurality of outer layers and changing the thickness of the coating layer as in one embodiment, but in the blood vessel 3a for practice, it is possible to implement the aneurysm inducing layer 25a generated at the specific position as in the actual blood vessel, such as forming the aneurysm inducing layer 25a by changing the components and strength of the outer layer as in another embodiment, thereby improving the proficiency of medical staff.

The spirit of the present invention has been described only by way of example hereinabove, and the present invention may be variously modified, altered, and substituted by those skilled in the art to which the present invention pertains without departing from essential features of the present invention. Accordingly, embodiments disclosed in the present invention and the accompanying drawings do not limit but describe the spirit of the present invention, and the scope of the present invention is not limited by the embodiments and the accompanying drawings. The scope of the present invention should be interpreted by the following claims, and it should be interpreted that all technical ideas equivalent to the following claims fall within the scope of the present invention.

### INDUSTRIAL APPLICABILITY

The present invention relates to an aneurysm prediction system using a deep learning algorithm, and the aneurysm prediction system is learned based on hemodynamic data calculated through a hemodynamic simulation, so that it is possible to accurately predict an aneurysm and minimize the time to predict the result. As a result, the present invention has high industrial applicability.

1. A method of augmenting aneurysm learning data, comprising:
performing a simulation using aneurysm data;
predicting a position having a smallest thickness in an aneurysm based on a result of the simulation;
setting a center position at the predicted position;
setting a plurality of peripheral positions at different positions having a preset radius from the center position;
extracting blood flow data according to a preset sampling period for a reference time at each of the center position and the plurality of peripheral positions;
converting the extracted blood flow data into an image to generate a blood flow image;
generating a central image and a peripheral image in which a plurality of blood flow images according to the center position and the peripheral position are arranged in an order of the reference time; and
generating different artificial images by changing an arrangement order of the central image and the peripheral image.

2. The method of claim 1, further comprising resetting a new peripheral position, which is different from the plurality of peripheral positions, from the center position.

3. The method of claim 2, wherein the resetting of the peripheral position includes setting at least one of a rotation direction from the center position, a rotation angle from the center position, the number of peripheral positions, and the radius to be different to set a new peripheral position located at a different position.

4. The method of claim 1, wherein the blood flow data includes at least one of blood flow velocity, pressure, a strain rate, a deformation amount, stress, force, wall shear stress (WSS), and an oscillatory shear index (OSI).

5. The method of claim 1, wherein, in the generating of the artificial image, a new artificial image is generated by combining the different artificial images according to different blood flow data.

6. The method of claim 1, wherein a size of the radius is changeable according to a size of the aneurysm.

7. The method of claim 1, wherein a size of the radius is changeable according to a degree of complexity of a shape of the aneurysm.

8. A method of augmenting aneurysm learning data, comprising:
performing a simulation using aneurysm data;
predicting a position having a smallest thickness in an aneurysm based on a result of the simulation;
setting a center position at the predicted position;
setting at least one of a rotation direction from the center position, a rotation angle from the center position, a radius from the center position, and a number to be different to set a plurality of peripheral positions spaced an equal distance from the center position in different directions;
extracting blood flow data according to a preset sampling period for a reference time at each of the center position and the plurality of peripheral positions;
converting the extracted blood flow data into an image to generate a blood flow image;
generating a central image and a peripheral image in which a plurality of blood flow images according to the center position and the peripheral position are arranged in an order of the reference time; and
generating an artificial image from the central image and the peripheral image.

9. The method of claim 8, further comprising, after the generating of the central image and the peripheral image, generating a new artificial image by changing an arrangement order of the central image and the peripheral image.

10. A system for augmenting aneurysm learning data, comprising:
a simulation module configured to perform a simulation using aneurysm data;
a positioning module configured to predict a position having a smallest thickness in an aneurysm based on a result of the simulation to set a center position at the predicted position and a plurality of peripheral positions at different positions having a preset radius from the center position;
a data extraction module configured to extract blood flow data according to a preset sampling period for a reference time at each of the center position and the plurality of peripheral positions; and
an artificial image generation module configured to convert the extracted blood flow data into an image to generate an artificial image.

11. The system of claim 10, wherein the positioning module resets a new peripheral position, which is different from the plurality of peripheral positions, from the center position.

12. The system of claim 10, wherein the artificial image generation module converts the blood flow data of the center position and a new peripheral position into an image to generate a blood flow image, and
combines a central image and a peripheral image in which a plurality of blood flow images according to the center position and the peripheral position are arranged in an order of the reference time to generate the artificial image.

13. The system of claim 12, wherein different artificial images are repeatedly generated by changing an arrangement order of the central image and the peripheral image.

14. The system of claim 11, wherein the positioning module sets a rotation direction from the center position, a rotation angle from the center position, the number of peripheral positions, and the radius to be different to set the new peripheral position located at a different position.

15. The system of claim 10, wherein the blood flow data includes at least one of blood flow velocity, pressure, a strain rate, a deformation amount, stress, force, wall shear stress (WSS), and an oscillatory shear index (OSI).

16. The system of claim 15, wherein the artificial image generation module generates a new artificial image by combining different artificial images according to different blood flow data.

17. The system of claim 10, wherein a size of the radius is changeable according to a size of the aneurysm.

18. The system of claim 10, wherein a size of the radius is changeable according to a degree of complexity of a shape of the aneurysm.
